(19) United States
(12) Patent Application Publication
(10) Pub. No.: US 2023/0165480 A1
(43) Pub. Date: Jun. 1, 2023

(54) APPARATUS AND METHOD FOR GENERATING VOLUME SELECTIVE THREE-DIMENSIONAL MAGNETIC RESONANCE IMAGE

(71) Applicant: RESEARCH & BUSINESS FOUNDATION SUNGKYUNKWAN UNIVERSITY, Suwon-si (KR)

(72) Inventors: Jang Yeon PARK, Suwon-si (KR); Jinil PARK, Suwon-si (KR)

(73) Assignee: RESEARCH & BUSINESS FOUNDATION SUNGKYUNKWAN UNIVERSITY, Suwon-si (KR)

(21) Appl. No.: 17/920,650

(22) PCT Filed: Mar. 8, 2021
(86) PCT No.: PCT/KR2021/002801

§ 371 Date: Oct. 21, 2022

(30) Foreign Application Priority Data
Apr. 22, 2020 (KR) 10-2020-0048957

(51) Int. Cl. A61B 5/055; A61B 5/00; G01R 33/565

(52) U.S. Cl. CPC A61B 5/055; A61B 5/0033; G01R 33/565

(57) ABSTRACT

An apparatus and a method for generating a volume-selective three-dimensional magnetic resonance image are disclosed. The volume-selective three-dimensional magnetic resonance image generating method according to an exemplary embodiment of the present disclosure includes applying a frequency selective excitation pulse and a slab selection gradient magnetic field together to an object; acquiring a signal generated from the object by the excitation pulse and the slab selection gradient magnetic field; and generating a three-dimensional magnetic resonance image through encoding based on a readout gradient magnetic field maintaining vertically to the acquired signal and the slab selection gradient magnetic field.

### TECHNICAL FIELD

The present disclosure relates to an apparatus and a method for generating a volume-selective three-dimensional magnetic resonance image.

### BACKGROUND ART

Generally, a magnetic resonance imaging (MRI) device acquires a tomographic image of a specific part of a patient using a resonance phenomenon occurred by supplying an electromagnetic energy and has an advantage that tomographic images are easily obtained as compared with imaging devices such as X-rays or CT without being exposed to radiation. Further, the MRI devices three-dimensionally show not only an anatomical structure of the body, but also various functional information at a desired angle to be widely used for accurate diagnosis of diseases.

Three-dimensional radial data acquisition magnetic resonance imaging techniques of the related art perform spin excitation of an object using non (frequency)-selective pulse or a selective pulse and sample free induction decay (FID) signals or echo signals along a plurality of trajectories on a three-dimensional data space (in other words, k-space) by means of the radial data acquisition method.

Various technological studies are conducted with regard to the magnetic resonance imaging technique and representatively, there is a UTE-MRI which is an imaging technique to implement an ultrashort echo time without using phase encoding.

The UTE imaging technique has recently received a lot of attention and is utilized in various fields because it may identify materials or tissues (for example, bones or ligaments) with a very short T2/T2* that is not seen from the magnetic resonance image of the related art.

However, the spin excitation method which is applied to the three-dimensional radial data acquisition method of the related art corresponds to a non-selective excitation method that the spin of the entire material is excited, rather than the slab (or volume) selective excitation. By doing this, data including not only information in a desired field of view (FOV), but also information out of the FOV is inevitably acquired. When the image is reconstructed with data including information out of the FOV, if the Nyquist condition is not satisfied, a streak artifact is generated in the image. Further, when the size of the object is larger than the field of view, this problem is more serious.

The streak artifact generated as described above causes a lot of errors not only during image processing tasks such as image segmentation and registration in a region to be analyzed, but also during clinical diagnosis, anatomical structure evaluation and voxel-wise quantitative analysis of images, which greatly undermines the meaning of the quantitative analysis itself.

For example, in the case of lung MRI which has recently received much attention as an alternative to CT which has a problem of high radiation dose, there is a serious problem due to the streak artifacts due to a signal generated from the tissues out of the field of view, such as a neck, an abdomen, and an arm.

A related art of the present disclosure is disclosed in Korean Registered Patent Publication No. 10-1775028.

### DISCLOSURE

### Technical Problem

In order to solve the above-described problems of the related art, an object of the present disclosure is to provide an apparatus and a method for generating a volume-selective three-dimensional magnetic resonance image which minimizes artifacts caused by signals generated from tissues out of the field of view during the magnetic resonance image generation based on three-dimensional radial data acquisition.

In order to solve the above-described problems of the related art, an object of the present disclosure is to provide an apparatus and a method for generating a volume-selective three-dimensional magnetic resonance image which minimizes the influence of the signal generated from the tissues out of the FOV by means of an encoding technique based on three-dimensional slab selective excitation and readout gradient magnetic field vertical to the slab selective gradient magnetic field, rather than exciting the entire object.

However, objects to be achieved by various embodiments of the present disclosure are not limited to the technical objects as described above and other technical objects may be present.

### Technical Solution

As a technical means to achieve the above-described technical object, according to an exemplary embodiment of the present disclosure, a volume-selective three-dimensional magnetic resonance image generating method may include: applying a frequency selective excitation pulse and a slab selection gradient magnetic field together to an object; acquiring a signal generated from the object by the excitation pulse and the slab selection gradient magnetic field; and generating a three-dimensional magnetic resonance image through encoding based on a readout gradient magnetic field maintaining vertically to the acquired signal and the slab selection gradient magnetic field.

Further, in the applying to the object, the slab selection gradient magnetic field which is determined to selectively spin-excite a predetermined volume region corresponding to a predetermined field of view may be applied to the object.

Further, in the applying to the object, an asymmetric frequency selective excitation pulse or a symmetric frequency selective excitation pulse may be applied.

Further, the asymmetric frequency selective excitation pulse may include a main lobe and include an asymmetric sinc pulse from which a side lobe following the main lobe is removed.

Further, the acquiring of a signal may include: acquiring an echo signal in a ramp period of the readout gradient magnetic field.

Further, the volume-selective three-dimensional magnetic resonance image generating method according to an exemplary embodiment of the present disclosure may include applying gridding interpolation to the acquired signal.

Further, the volume-selective three-dimensional magnetic resonance image generating method according to an exemplary embodiment of the present disclosure may include selecting a predetermined volume region based on a field of view corresponding to a region of the object to be captured; and determining the slab selection gradient magnetic field corresponding to the selected volume region.

Further, in the applying to the object, a plurality of slab selection gradient magnetic fields having a slab selection direction corresponding to each axial direction of the three-dimensional coordinate system may be applied to the object.

Further, in the generating of a magnetic resonance image, the encoding may be performed based on a plurality of readout gradient magnetic fields applied to each axial direction of the three-dimensional coordinate system so as to vertically correspond to the plurality of slab selection magnetic fields.

Further, the frequency encoding method using the readout gradient magnetic field perpendicular to the direction of the slab selection gradient magnetic field includes various methods such as a spiral type.

In the meantime, according to an exemplary embodiment of the present disclosure, a volume-selective three-dimensional magnetic resonance image generating apparatus may include an excitation execution unit which applies a frequency selective excitation pulse and a slab selection gradient magnetic field together to an object; a data acquisition unit which acquires a signal generated from the object by the excitation pulse and the slab selection gradient magnetic field; and an encoding unit which generates a three-dimensional magnetic resonance image through encoding based on a readout gradient magnetic field maintaining vertically to the acquired signal and the slab selection gradient magnetic field.

Further, the excitation execution unit may apply the slab selection gradient magnetic field which is determined to selectively spin-excite a predetermined volume region corresponding to a predetermined field of view to the object.

Further, the excitation execution unit may apply an asymmetric frequency selective excitation pulse or a symmetric frequency selective excitation pulse.

Further, the asymmetric frequency selective excitation pulse may include a main lobe and include an asymmetric sinc pulse from which a side lobe following the main lobe is removed.

The data acquisition unit may acquire an echo signal which is encoded by the readout gradient magnetic field perpendicular to the slab selection gradient magnetic field in a ramp period of the readout gradient magnetic field.

Further, the volume-selective three-dimensional magnetic resonance image generating apparatus according to an exemplary embodiment of the present disclosure may include a region setting unit which selects a predetermined volume region based on a field of view corresponding to a region of the object to be captured and determines the slab selection gradient magnetic field corresponding to the selected volume region.

The above-described solving means are merely illustrative but should not be construed as limiting the present disclosure. In addition to the above-described embodiments, additional embodiments may be further provided in the drawings and the detailed description of the present disclosure.

### Advantageous Effects

According to the above-described solving means of the present disclosure, it is possible to provide an apparatus and a method for generating a volume-selective three-dimensional magnetic resonance image which minimizes artifacts caused by signals generated from tissues out of the field of view during the magnetic resonance image generation based on three-dimensional radial data acquisition.

According to the above-described solving means of the present disclosure, it is possible to provide a volume-selective three-dimensional magnetic resonance image generating apparatus and method which minimize the influence of the signal generated from the tissue out of the FOV by three-dimensional slab selective excitation, rather than excitation of the entire object.

According to the above-described solving means of the present disclosure, the slab gradient magnetic field is designed to be vertical to the readout gradient magnetic field at all times so that the region out of the field of view which is not excited by the spin excitation in the slab selection direction is not included during data acquisition, thereby effectively selecting a three-dimensional volume.

According to the above-described solving means of the present disclosure, the FOV is set to be close to the region of interest (ROI) as much as possible so that a higher resolution image may be obtained during the same scanning time and an image showing an equal level of resolution for a shorter scanning time may be acquired.

However, the effect which can be achieved by the present disclosure is not limited to the above-described effects, and there may be other effects.

### DESCRIPTION OF DRAWINGS

FIG. 1 is a schematic diagram of an MRI system including a volume-selective three-dimensional magnetic resonance image generating apparatus according to an exemplary embodiment of the present disclosure.

FIG. 2 is a view illustrating an excitation pulse and a gradient magnetic field applied to generate a volume-selective three-dimensional magnetic resonance image according to an exemplary embodiment of the present disclosure compared with the UTE technique of the related art.

FIG. 3 is a conceptual view for explaining that a predetermined volume region of an object is selected by a frequency selective excitation pulse and a slab selection gradient magnetic field applied according to an exemplary embodiment of the present disclosure and that an echo signal encoded by a readout gradient magnetic field vertical to the slab selection gradient magnetic field does not include information of an object which is not selected as a volume region.

FIG. 4 is a view illustrating a cross-sectional plane and a coronal plane of a phantom captured by each of the UTE technique of the related art and a volume-selective three-dimensional magnetic resonance image generating technique of the present disclosure, as an experimental example associated with the volume-selective three-dimensional magnetic resonance image generating technique according to an exemplary embodiment of the present disclosure.

FIG. 5 is a view illustrating a cross-sectional plane and a coronal plane of a lung of a healthy subject captured by each of the UTE technique of the related art and a volume-selective three-dimensional magnetic resonance image generating technique of the present disclosure, as an experimental example associated with the volume-selective three-dimensional magnetic resonance image generating technique according to an exemplary embodiment of the present disclosure.

FIG. 6 is an operation flowchart of a volume-selective three-dimensional magnetic resonance image generating method according to an exemplary embodiment of the present disclosure.

### BEST MODE

Hereinafter, the present disclosure will be described more fully hereinafter with reference to the accompanying drawings, in which exemplary embodiments of the present disclosure are shown so that those of ordinary skill in the art to which the present disclosure pertains can easily carry out. However, the present disclosure can be realized in various different forms, and is not limited to the embodiments described herein. Accordingly, in order to clearly explain the present disclosure in the drawings, portions not related to the description are omitted. Like reference numerals designate like elements throughout the specification.

Throughout this specification and the claims that follow, when it is described that an element is “coupled” to another element, the element may be “directly coupled” to the other element or “electrically coupled” or “indirectly coupled” to the other element through a third element.

Through the specification of the present disclosure, when one member is located “on”, “above”, “on an upper portion”, “below”, “under”, and “on a lower portion” of the other member, the member may be adjacent to the other member or a third member may be disposed between the above two members.

In the specification of the present disclosure, unless explicitly described to the contrary, the word “comprise” and variations such as “comprises” or “comprising”, will be understood to imply the inclusion of stated elements but not the exclusion of any other elements.

The present disclosure relates to an apparatus and a method for generating a volume-selective three-dimensional magnetic resonance image.

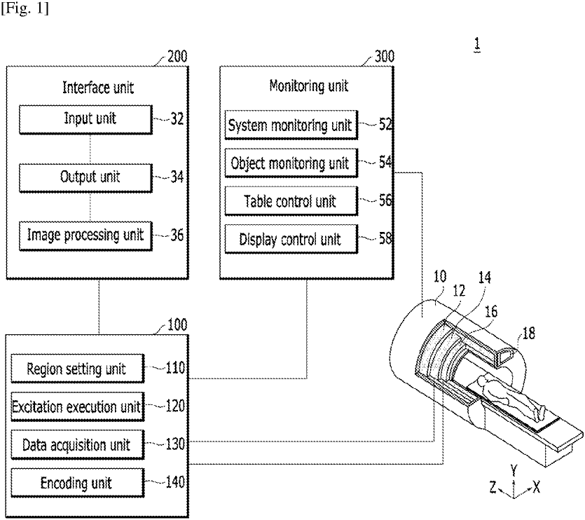

FIG. 1 is a schematic diagram of an MRI system including a volume-selective three-dimensional magnetic resonance image generating apparatus according to an exemplary embodiment of the present disclosure.

Referring to FIG. 1, an MRI system 1 according to an exemplary embodiment of the present disclosure may include an MRI scanner 10, a volume-selective three-dimensional magnetic resonance image generating apparatus 100 according to an exemplary embodiment of the present disclosure (hereinafter, simply referred to as “magnetic resonance image generating apparatus 100”), an interface unit 200, and a monitoring unit 300. For reference, in the description of an exemplary embodiment of the present disclosure, a magnetic resonance imaging (MRI) system 1 comprehensively refers to a system which applies a magnetic field and a specific ionization radiation (radio frequency) to an object to acquire an image based on a physical principle, such as nuclear magnetic resonance (NMR).

First, the MRI scanner 10 forms a magnetic field, generates a resonance phenomenon for atomic nuclei, and a magnetic resonance image is captured in a state where the object is located in the MRI scanner 10. The MRI scanner 10 may include a main magnet 12, a gradient coil 14, and an RF coil 16 and form a static magnetic field and a gradient magnetic field thereby, and irradiate an RF signal onto the object. For example, in the description of the exemplary embodiment of the present disclosure, an object refers to a target to be imaged by the MRI system 1 and may include humans or animals or a part of them. Further, the object may include various organs such as heart, brain, or blood vessels or various phantoms.

The main magnet 12, the gradient coil 14, and the RF coil 16 are disposed in the MRI scanner 10 along a predetermined direction. The object is located on a table which is inserted into a cylinder along a horizontal axis of the cylinder and the object may be located in a bore of the MRI scanner 10 according to the movement of the table.

The main magnet 12 may generate a static magnetic field which aligns a direction of magnetic dipole moments of atomic nucleus included in the object in a predetermined direction.

The gradient coil 14 includes an X coil, a Y coil, and a Z coil which generate gradient magnetic fields in X-axis, Y-axis, and Z-axis directions perpendicular to one another. The gradient coil 14 induces a different resonance frequency for every part of the object to obtain position information of each part of the object.

The RF coil 16 may irradiate an RF signal to the object and receive a magnetic resonance image signal emitted from the object. The RF coil 16 may output the RF signal having the same frequency as a frequency of a processional motion to the atomic nuclei which performs the processional motion and then receive the magnetic resonance image signal emitted from the object.

For example, the RF coil 16 generates the RF signal having a frequency corresponding to the atomic nuclei and applies the RF signal to the object to shift the atomic nuclei from a low energy state to a high energy state. Thereafter, when the RF coil 16 stops transmitting the RF signal, the atomic nucleus to which an electromagnetic wave is applied emits an electromagnetic wave having a Lamor frequency while shifting from the high energy state to the low energy state and the RF coil 16 may receive the corresponding electromagnetic wave signal.

The RF coil 16 may include a transmission RF coil which transmits an RF signal having a radio frequency corresponding to a type of the atomic nucleus and a reception RF coil which receives an electromagnetic wave emitted from the atomic nucleus.

Further, the RF coil 16 may be fixed to the MRI scanner 10 or may be detachable from the MRI scanner. The detachable RF coil 16 may be implemented by a head RF coil, a chest RF coil, a leg RF coil, a neck RF coil, a shoulder RF coil, a wrist RF coil, and an ankle RF coil which are coupled to a part of the object.

The MRI scanner 10 may include a display 18 which provides various information to a user or the object. According to an implemented example of the present disclosure, as illustrated in FIG. 1, the display 18 may be disposed at the outside of the MRI scanner 10, but is not limited thereto. As another example, the display 18 may be disposed in the MRI scanner 10. For reference, in the description of the exemplary embodiment of the present disclosure, the user may be a medical expert such as a doctor, a nurse, or a medical imaging specialist, or a device repairing engineer, but is not limited thereto.

The interface unit 30 may transmit instructions about pulse sequence information according to the manipulation of the user and transmit a command to control an overall operation of the MRI system 1. The interface unit 30 may include an image processing unit 36 which processes a magnetic resonance image signal received from the MRI scanner 10, an output unit 34, and an input unit 32. The image processing unit 36 processes the magnetic resonance image signal received from the MRI scanner 10 to generate MR image data for the object.

Further, the image processing unit 36 may apply various signal processing, such as amplifying, frequency converting, phase detecting, low-frequency amplifying, and filtering, to the magnetic resonance image signal received from the MRI scanner 10.

Further, according to the exemplary embodiment of the present disclosure, the image processing unit 36 may operate to dispose digital data in a k-space and perform two-dimensional or three-dimensional Fourier conversion on the data to be reconstructed as image data.

Further, various signal processing applied by the image processing unit 36 to the magnetic resonance image signal received from the MRI scanner 10 may be performed in parallel. For example, the signal processing is applied to a plurality of magnetic resonance image signals received by multi-channel RF coil in parallel to reconstruct the plurality of magnetic resonance image signals as image data.

The output unit 34 may output image data or reconstructed image data generated by the image processing unit 36 to the user. Further, the output unit 34 may output information required for the user to manipulate the MRI system, such as user interface (UI), user information, or object information. The output unit 34 may include a speaker, a printer, or various image display units.

The user may input object information, parameter information, a scan condition, a pulse sequence, and information about image composition or operation of difference by means of the input unit 32. The input unit 32 may include a keyboard, a mouse, a track ball, a voice recognition unit, a gesture recognition unit, and a touch screen and include various input devices within a range apparent to those skilled in the art.

The monitoring unit 50 monitors or controls the MRI scanner 10 or devices mounted in the MRI scanner 10. The monitoring unit 50 may include a system monitoring unit 52, an object monitoring unit 54, a table control unit 56, and a display control unit 58.

The system monitoring unit 52 may monitor and control a state of the static magnetic field, a state of a gradient magnetic field, a state of an RF signal, a state of an RF coil, a state of the table, a state of a device which measures body information of the object, a power supply state, a state of a heat exchanger, or a state of a compressor.

The object monitoring unit 54 monitors the condition of the object and may include a camera which captures a motion or a position of the object, a respiration meter which measures respiration of the object, an ECG meter which measures an electrocardiogram of the object, or a body temperature measurement unit which measures a temperature of the object.

The table control unit 56 may control the movement of the table on which the object is located. For example, the table control unit 56 may move the table in response to the sequence control for moving imaging of the subject and thus capture the object with a field of view (FOV) larger than the field of view (FOV) of the MRI scanner 10.

The display control unit 58 may control the display disposed outside and inside of the MRI scanner 10 to be on/off and control the screen to be output to the display. Further, when the speaker is located inside or outside the MRI scanner 10, the display control unit 58 may control the speaker to be on/off or control a sound to be output through the speaker.

The MRI scanner 10, the magnetic resonance image generating apparatus 100, the interface unit 200, and the monitoring unit 300 may communicate with each other via a network (not illustrated). The network (not illustrated) means a connection structure which allows information exchange between nodes such as terminals or servers. Examples of the network (not illustrated) include 3^{rd} generation partnership project (3GPP) network, a long term evolution network, a 5G network, a world interoperability for microwave access (WIMAX) network, Internet, a local area network (LAN), a wireless local area network (Wireless LAN), a wide area network (WAN), a personal area network (PAN), a Wi-Fi network, a Bluetooth network, a satellite broadcasting network, an analog broadcasting network, and a digital multimedia broadcasting (DMB) network, but are not limited thereto. That is, the MRI scanner 10, the magnetic resonance image generating apparatus 100, the interface unit 200, and the monitoring unit 300 may be connected to each other in a wireless or wired manner. When the units are connected wirelessly, a device (not illustrated) for synchronizing clocks therebetween may be included. As another example, as the communication between the MRI scanner 10, the magnetic resonance image generating apparatus 100, the interface unit 200, and the monitoring unit 300, a high speed digital interface such as low voltage differential signaling (LVDS), asynchronous serial communication such as universal asynchronous receiver transmitter (UART), error-synchronous serial communication, or low latency network protocol such as controller area network (CAN), optical communication may be used, and various communication methods may be used within a range apparent to those skilled in the art.

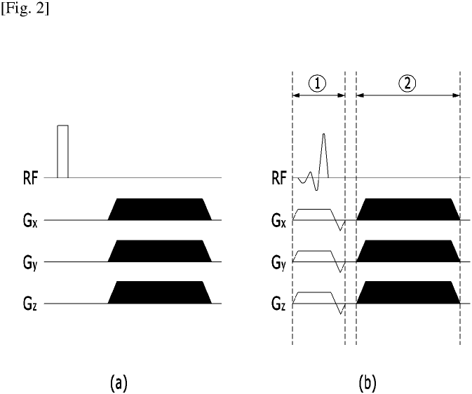

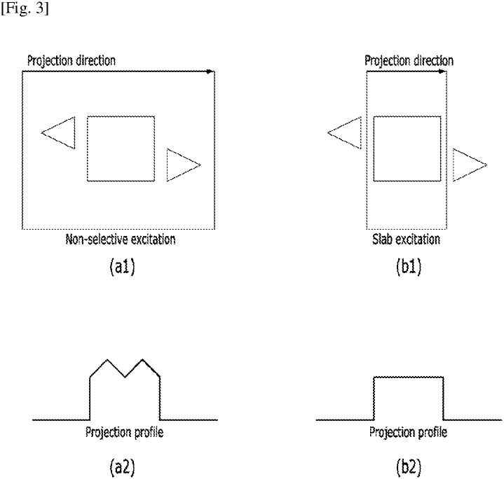

Hereinafter, a specific function and operation of the magnetic resonance image generating apparatus 100 disclosed in the present disclosure will be described in more detail with reference to FIGS. 2 and 3.

The magnetic resonance image generating apparatus 100 may control the gradient magnetic field formed in the MRI scanner 10 according to a predetermined MR pulse sequence (that is, a pulse sequence) and control transmission/reception of the RF signal and the magnetic resonance image signal which are excitation pulses.

Further, the magnetic resonance image generating apparatus 100 may drive the gradient coil 14 included in the MRI scanner 10 and supply a signal which generates the gradient magnetic field to the gradient coil 14. With regard to this, the magnetic resonance image generating apparatus 100 controls a pulse signal supplied to the gradient coil 14 from a gradient magnetic field amplifier to compose gradient magnetic fields in X-axis, Y-axis, and Z-axis directions.

Further, the magnetic resonance image generating apparatus 100 supplies the RF pulse to the RF coil 16 to drive the RF coil 16. Further, the magnetic resonance image generating apparatus 100 may receive the magnetic resonance image signal which is received by the RF coil 16 and then transmitted thereto. For reference, the magnetic resonance image signal received by the magnetic resonance image generating apparatus 100 may be a free induction decay (FID) signal or an echo signal.

Further, the magnetic resonance image generating apparatus 100 may adjust a transmission/reception direction of the RF signal and the magnetic resonance image signal. For example, the magnetic resonance image generating apparatus 100 may irradiate the RF signal to the object by means of the RF coil 16 during the transmission operation and receive the magnetic resonance image signal from the object by means of the RF coil 16 during the reception operation.

Further, referring to FIG. 1, the magnetic resonance image generating apparatus 100 may include a region setting unit 110, an excitation execution unit 120, a data acquisition unit 130, and an encoding unit 140.

The region setting unit 110 may select a predetermined volume region for the object based on a field of view (FOV) corresponding to a region of the object to be captured. In order to acquire a lung region, for example, the region setting unit 110 may set the field of view (FOV) with respect to the lung, but set the field of view FOV so as not to include tissues or organs outside the lung, such as the arm, the abdomen, and the neck. With regard to this, the excitation execution unit 120 to be described below may determine (compose) a slab selection gradient magnetic field corresponding to the selected volume region.

FIG. 2 is a view illustrating an excitation pulse and a gradient magnetic field applied to generate a volume-selective three-dimensional magnetic resonance image according to an exemplary embodiment of the present disclosure compared with the UTE technique of the related art.

Specifically, (a) of FIG. 2 illustrates a pulse sequence applied according to the ultrashort echo time (UTE) technique of the related art and (b) of FIG. 2 illustrates a pulse sequence applied according to the volume-selective three-dimensional magnetic resonance image generating technique disclosed in the present disclosure.

Referring to (b) of FIG. 2, the excitation execution unit 120 may apply the frequency selective excitation pulse (RF of FIG. 2) and slab selection gradient magnetic fields Gₓ, G_{y}, and G_{z} of FIG. 2 together to the object (1). Here, the excitation execution unit 120 may apply the slab selection gradient magnetic fields determined to selectively spin-excite a predetermined volume region of the object corresponding to a predetermined field of view (FOV), rather than the entire region of the object.

Further, according to the exemplary embodiment of the present disclosure, the excitation execution unit 120 may apply a plurality of slab selection gradient magnetic fields having slab selection directions corresponding to individual axial directions (in other words, an X-axis direction, a Y-axis direction, and a Z-axis direction) of a three-dimensional coordinate system to the object to selectively excite the three-dimensionally formed volume region. In other words, the excitation execution unit 120 may excite only a region (selected volume region) from which the user wants to have a magnetic resonance image, from a region which forms the object, by means of the plurality of slab selection gradient magnetic fields with respect to the axial directions which is applied together with the frequency selective excitation pulse.

With regard to this, in the related citation [Bergin C, Pauly J, Macovski A. “Lung parenchyma projection reconstruction MR imaging.” Radiology 1991; 179(3) 777 to 781.], a method of encoding a free induction decay signal in a three-dimensional k-space by spin-exciting the entire object using a frequency non-selective pulse and acquiring radiation data has been disclosed. According to the method disclosed in the related citation, a design of the pulse sequence was simple to be easily implemented. However, in the related citation, the entire object was spin-excited so that unintended information out of the field of view was encoded together with the data so that there was a problem in that streak artifacts may be caused by the under-sampling. Further, a free induction decay signal was acquired rather than the echo signal so that there was a limitation in that the middle region (center) of the k-space may be lost during the sampling.

Further, in the related art citation [Johnson KM, Fain SB, Schiebler ML, Nagle S. “Optimized 3D ultrashort echo time pulmonary MRI.” Magnetic resonance in medicine 2013; 70(5): 1241 to 1250.], a method of performing spin-excitation by applying a frequency-selective pulse and a slab selection gradient magnetic field fixed to the Z-axis together and sampling a three-dimensional k-space has been disclosed as a three-dimensional radial magnetic resonance imaging technique using a frequency selective pulse. In the above-described document, the slab selective spin excitation fixed to the Z-axis was performed so that only information in the field of view was acquired in the Z-axis, but there was a limitation in that unintended information out of the field of view was still acquired from the X-axis and the Y-axis.

As described above, some of the three-dimensional radial data acquisition magnetic resonance imaging techniques have employed a method of performing slab-selective spin excitation by utilizing a frequency selective pulse and a gradient magnetic field. However, the selective spin excitation was never allowed in the direction perpendicular to the slab gradient magnetic field so that it was not possible to substantially select a three-dimensional volume corresponding to the field of view. In contrast, the magnetic resonance image generating apparatus 100 disclosed in the present disclosure selects the three-dimensional volume when the radial data is acquired to minimize the streak artifacts of the image caused by the object out of the field of view. Specifically, for example, in order to acquire a lung image, the region setting unit 110 sets the field of view (FOV) with respect to the lung so that signals generated from the arm, the abdomen, and the neck out of the field of view are excluded when the data is acquired. By doing this, the artifacts caused by the signal generated from the object out of the field of view are effectively reduced to improve a quality of the image and enable the accurate clinical diagnosis and quantitative image analysis for every voxel.

Further, the excitation execution unit 120 may apply a symmetric frequency selective excitation pulse or an asymmetric frequency selective excitation pulse. Further, referring to (b) of FIG. 2, according to the exemplary embodiment of the present disclosure, the asymmetric frequency selective excitation pulse applied by the excitation execution unit 120 includes a main lobe and may include an asymmetric sinc pulse from which a side lobe following the main lobe is removed. However, a pulse type of the frequency selective excitation pulse applied by the excitation execution unit 120 is not limited only to the sinc pulse, and according to the implemented example of the present disclosure, all types of frequency selective execution pulses such as a pulse made by an SLR algorithm and a modified sinc pulse may be broadly applied.

Specifically, with regard to excitation pulse applied by the excitation execution unit 120, contrary to the magnetic resonance imaging technique of the related art which generally uses a sinc function type symmetric pulse which is a frequency selective pulse, in an imaging technique for achieving a very short echo time, such as a UTE imaging technique, an asymmetric excitation pulse is applied to shorten the echo time.

That is, when a symmetric frequency selective pulse which is mainly used in the related art and the slab selective gradient magnetic field are used together, it is necessary to apply a gradient magnetic field having an opposite polarity to rephrase spins which are dephased in a slab direction by the slab selection gradient magnetic field as much as half an area of the gradient magnetic field used for spin excitation, while the pulse ends from the center of the pulse. However, it acts as a constraint in the UTE image which needs to reduce the echo time as much as possible. In contrast, when the asymmetric frequency selective pulse is used, it is advantageous in that the dephased area itself is reduced to effectively shorten the echo time.

Further, the data acquisition unit 130 may acquire a signal generated from the object by the excitation pulse applied to the object and the slab selection gradient magnetic field. According to the exemplary embodiment of the present disclosure, a signal generated from the object may refer to the echo signal, but it is not limited thereto so that according to the implemented example of the present disclosure, a signal generated from the object may include a free induction decay signal.

According to the exemplary embodiment of the present disclosure, the data acquisition unit 130 may acquire an echo signal which is encoded by the readout gradient magnetic field perpendicular to the slab selection gradient magnetic field during a ramp period of the readout gradient magnetic field. Further, according to the exemplary embodiment of the present disclosure, the echo signal acquired by the data acquisition unit 130 may be an asymmetric echo signal, but a type of the echo signal is not limited thereto.

As another example, the data acquisition unit 130 according to an exemplary embodiment of the present disclosure may operate to acquire an echo signal when a magnitude of the gradient magnetic field reaches a predetermined level (for example, a predetermined magnitude) after passing the ramp period.

With regard to the echo signal acquisition timing of the data acquisition unit 130, according to the UTE magnetic resonance imaging technique of the related art, in order to acquire a very short echo time, the free induction decay (FID) signal is generally acquired after the spin excitation. In this case, it is highly likely to lose samples corresponding to the center portion of the k-space so that it is difficult to obtain a stable image. In contrast, when the asymmetrical echo signal is acquired instead of the free induction decay (FID) signal, like the data acquisition unit 130 of the present disclosure, there is an advantage in that it is possible to obtain a stable image because there is a relatively little chance of not being able to sample the center of the k-space. In other words, the inventor of the present disclosure wants to ensure a quality of a stable image and a very short echo time by obtaining an asymmetrical echo signal in the ramp period of the gradient magnetic field.

Specifically, when the asymmetrical echo signal is acquired rather than the free induction decay (FID) signal after spin excitation, it is robust to the error caused by an eddy current due to the gradient magnetic field and the time delay so that it is possible to more stably acquire the data. Specifically, the echo signal is acquired when the readout gradient magnetic field reaches a predetermined magnitude. In order to shorten the time to reach the predetermined magnitude of the readout gradient magnetic field, the data acquisition unit 130 may acquire an asymmetric echo signal in the ramp period of the readout gradient magnetic field. The echo signal is acquired as described above so that even though the echo signal is asymmetric, an echo peak corresponding to the center of the k-space is acquired so that an image stable more than the free induction decay (FID) signal may be obtained.

Further, according to the exemplary embodiment of the present disclosure, the data acquisition unit 130 may determine an acquisition timing of the asymmetrical echo signal based on an area of the entire gradient magnetic field (the slab selection gradient magnetic field and the readout gradient magnetic field) more precisely in the ramp period of the readout gradient magnetic field.

Further, referring to (b) of FIG. 2, the encoding unit 140 may generate a three-dimensional magnetic resonance image by encoding based on the readout gradient magnetic field maintaining vertically to the acquired echo signal and the slab selection gradient magnetic field (2). Here, when the readout gradient magnetic field maintains vertically to the slab selection gradient magnetic field, it is understood that a slab selection direction for spin excitation and a data acquisition direction (that is, a projected direction) for encoding are set to be equal.

Further, according to the exemplary embodiment of the present disclosure, the encoding unit 140 may perform the encoding based on the plurality of readout gradient magnetic fields applied toward the axial directions (in other words, the X-axis direction, the Y-axis direction, and the Z-axis direction) in the three-dimensional coordinate system so as to correspond to the plurality of slab selection gradient magnetic fields.

With regard to this, in the related art citation [Park JY, Moeller S, Goerke U, Auerbach E, Garwood M, et al. “Short echo-time 3D radial gradient-echo MRI using concurrent dephasing and excitation.” Magnetic resonance in medicine 2012; 67(2): 428 to 436.], a technique of performing spin excitation by applying a frequency selective pulse and a slab selection gradient magnetic field and acquiring an echo signal by applying a readout gradient magnetic field in an opposite direction to the slab selection gradient magnetic field has been disclosed. However, the echo signal was obtained by the readout gradient magnetic field which was applied in an opposite direction to the slab selection gradient magnetic field so that there was a limitation in that information out of the field of view was still reflected.

Further, in the related art citation [Weiger M, Pruessmann KP, Hennel F. “MRI with zero echo time: hard versus sweep pulse excitation.” Magnetic resonance in medicine, 2011; 66(2): 379 to 389], a method of sampling a signal after spin-excitation of an object using a non-selective pulse in a state in which the readout gradient magnetic field for radial data acquisition method used for the three-dimensional k-space sampling is applied has been disclosed. According to this method, the same effect as the excitation of the entire object was shown by the very short pulse length (for example, 5 us or shorter). Further, in the state in which the readout gradient magnetic field is applied, all the processes including the sampling were performed so that it showed a more effective result for a signal having a very short T2* than the UTE. However, the pulse was applied together with the readout gradient magnetic field so that there was a limitation in that it was not possible to set a long echo signal. Further, a time to apply a pulse and sample the data needs to be very short so that it was disadvantageous in that a high performance hardware is necessary.

In contrast, the magnetic resonance image generating apparatus 100 according to the exemplary embodiment of the present disclosure is designed such that the slab gradient magnetic field and the readout gradient magnetic field are always formed to be vertical to be applied to the object. By doing this, an area which is not excited by the spin excitation in the slab selection direction is not included during the data acquisition so that the three-dimension volume may be effectively selected.

That is, the encoding unit 140 according to the exemplary embodiment of the present disclosure applies a direction of the readout gradient magnetic field for k-space encoding to generate the magnetic resonance image to be vertical to the slab selection gradient magnetic field so that data is obtained by the projection in the slab direction. By doing this, the object out of the field of view on which the spin excitation is not performed may not be influenced. In other words, the encoding unit 140 may perform the encoding while maintaining the slab selection gradient magnetic field which changes along the direction of the trajectory to fill the three-dimensional k-space and the readout gradient magnetic field vertical thereto.

Further, according to the exemplary embodiment of the present disclosure, the magnetic resonance image generating apparatus 100 may apply gridding interpolation to the acquired asymmetric echo signal.

FIG. 3 is a conceptual view for explaining that a predetermined volume region of an object is selected by a frequency selective excitation pulse and a slab selection gradient magnetic field applied according to an exemplary embodiment of the present disclosure and that an echo signal encoded by a readout gradient magnetic field vertical to the slab selection gradient magnetic field does not include information of an object which is not selected as a volume region.

Specifically, (a1) and (a2) of FIG. 3 illustrate that a region other than a volume region corresponding to a set field of view selected by the UTE imaging technique of the related art affects data to be acquired and (b1) and (b2) of FIG. 3 illustrate that a signal by the object out of the field of view does not affect the data to be acquired by the volume selection according to the volume-selective three-dimensional magnetic resonance image generating technique disclosed in the present disclosure.

Referring to FIG. 3, according to the UTE imaging technique of the related art, a spin from the entire region of the object affects the generated signal (see (a2) of FIG. 3), but according to the volume-selective three-dimensional magnetic resonance image generating technique disclosed in the present disclosure, it is confirmed that the frequency-selective excitation pulse is applied to the object together with the slab selection gradient magnetic field vertical to the readout gradient magnetic field so that the signal generated from the object out of the field of view (FOV) (or out of the region of interest (ROI)) may be effectively suppressed (see (b2) of FIG. 3).

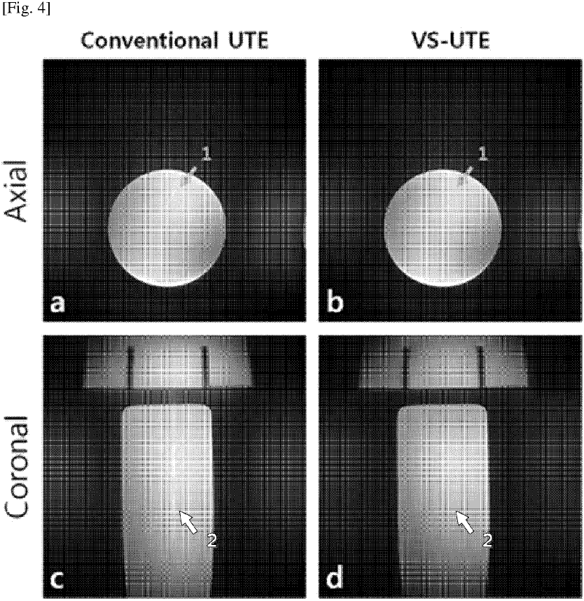

FIG. 4 is a view illustrating a cross-sectional plane and a coronal plane of a phantom captured by each of the UTE technique of the related art and a volume-selective three-dimensional magnetic resonance image generating technique of the present disclosure as an experimental example associated with the volume-selective three-dimensional magnetic resonance image generating technique according to an exemplary embodiment of the present disclosure.

For reference, an experiment using a phantom illustrated in FIG. 4 was performed on an object in which an ACR phantom, two 1900 ml-saline solution phantoms (model No. 8624186) by Siemens, and a 5300 ml-cylindrical water phantom (model No. 10606530) by Siemens were disposed so as to correspond to the positions of the neck, the arm, and the body, respectively. Further, the field of view (FOV) for the object was set such that predetermined regions of a phantom corresponding to the arm and a phantom corresponding to the body corresponding to a part below the abdomen were not included.

With regard to this, referring to FIG. 4, it is confirmed that the streak artifacts generated in the image due to the signal generated in the region out of the set field of view (FOV) are generated in the conventional UTE image which uses the frequency non-selective pulse (for example, a rectangular pulse) (see FIGS. 4A and 4C), but the streak artifacts are hardly generated by the volume-selective three-dimensional magnetic resonance image generating technique disclosed in the present disclosure (FIGS. 4B and 4D).

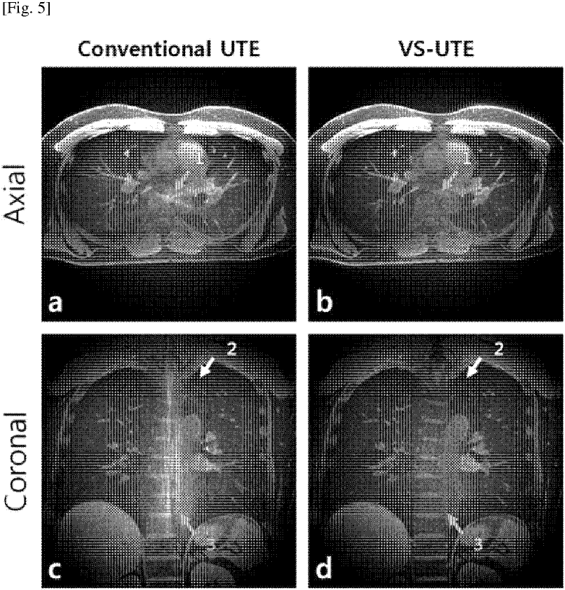

FIG. 5 is a view illustrating a cross-sectional plane and a coronal plane of a lung of a healthy subject captured by each of the UTE technique of the related art and a volume-selective three-dimensional magnetic resonance image generating technique of the present disclosure as an experimental example associated with the volume-selective three-dimensional magnetic resonance image generating technique according to an exemplary embodiment of the present disclosure.

In the experiment illustrated in FIG. 5, the field of view FOV is set to include only the lung region to minimize the influence of the signals generated from the neck, the abdomen (body), and the arm. Referring to FIG. 5, it is confirmed that in the conventional UTE image, the streak artifacts caused by the signal entering from the outside of the set field of view (FOV) such as the neck or the abdomen were observed (see FIGS. 5A and 5C), but according to the volume-selective three-dimensional magnetic resonance image generating technique disclosed in the present disclosure, this problem hardly occurs (see FIGS. 5B and 5D).

For reference, in the above description, the volume-selective three-dimensional magnetic resonance image generating technique of the present disclosure has been described for radial data acquisition or radial encoding, but it is not limited thereto and as another example, it may be connected to a helical encoding technique, and the like. Further, the present disclosure may be applied to be connected to a compressed sensing technique or a deep learning technique.

Hereinafter, an operation flow of the present disclosure will be described in brief based on the above-detailed description.

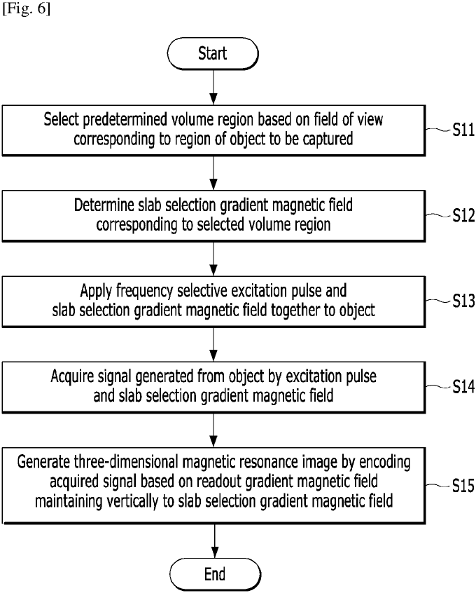

FIG. 6 is an operation flowchart of a volume-selective three-dimensional magnetic resonance image generating method according to an exemplary embodiment of the present disclosure.

The three-dimensional magnetic resonance image generating method illustrated in FIG. 6 may be performed by the above-described magnetic resonance image generating apparatus 100. Accordingly, even though contents are omitted below, if the contents have been described for the magnetic resonance image generating apparatus 100, the contents may also be applied to the description of the three-dimensional magnetic resonance image generating method in the same way.

Referring to FIG. 6, in step S11, the region setting unit 110 may select a predetermined volume region based on a field of view corresponding to a region of an object to be captured.

Next, in step S12, the excitation execution unit 120 may determine a slab selection gradient magnetic field corresponding to the selected volume region.

Next, in step S13, the excitation execution unit 120 may apply a frequency selective excitation pulse and a slab selection gradient magnetic field together to the object.

Further, in step S13, the excitation execution unit 120 may apply the slab selection gradient magnetic field determined to selectively spin-excite a predetermined volume region corresponding to the predetermined field of view to the object.

Further, in step S13, the excitation execution unit 120 may apply a symmetric frequency selective excitation pulse or an asymmetric frequency selective excitation pulse. Here, the asymmetric frequency selective excitation pulse includes a main lobe according to the exemplary embodiment of the present disclosure and may include an asymmetric sinc pulse from which a side lobe following the main lobe is removed.

Further, in step S13, the excitation execution unit 120 may apply a plurality of slab selection gradient magnetic fields having a slab selection direction corresponding to each axial direction of the three-dimensional coordinate system to the object.

Next, in step S14, the data acquisition unit 130 may acquire a signal generated from the object by the excitation pulse and the slab selective gradient magnetic field applied to the object.

Further, in step S14, the data acquisition unit 130 may acquire an echo signal in the ramp period of the readout gradient magnetic field.

Next, in step S15, the encoding unit 140 may generate a three-dimensional magnetic resonance image by encoding based on the signal acquired from the object and the readout gradient magnetic field maintaining vertically to the slab selection gradient magnetic field.

Further, in step S15, the encoding unit 140 may encode based on the plurality of readout gradient magnetic fields applied in the respective axial direction of the three-dimensional coordinate system so as to correspond to the plurality of slab selection gradient magnetic fields.

In the above-description, steps S11 to S15 may be further divided into additional steps or combined as smaller steps depending on an implementation example of the present disclosure. Further, some steps may be omitted if necessary and the order of steps may be changed.

The volume-selective three-dimensional magnetic resonance image generating method according to the embodiment of the present disclosure may be implemented as program instructions which may be executed by various computer means to be recorded in a computer readable medium. The computer readable medium may include solely a program command, a data file, and a data structure or a combination thereof. The program instruction recorded in the medium may be specifically designed or constructed for the present disclosure or known to those skilled in the art of a computer software to be used. Examples of the computer readable recording medium include magnetic media such as a hard disk, a floppy disk, or a magnetic tape, optical media such as a CD-ROM or a DVD, magneto-optical media such as a floptical disk, and a hardware device which is specifically configured to store and execute the program command such as a ROM, a RAM, and a flash memory. Examples of the program command include not only a machine language code which is created by a compiler but also a high level language code which may be executed by a computer using an interpreter. The hardware device may operate as one or more software modules in order to perform the operation of the present disclosure and vice versa.

Further, the above-described volume-selective three-dimensional magnetic resonance image generating method may also be implemented as a computer program or an application executed by a computer which is stored in a recording medium.

The above description of the present disclosure is illustrative only and it is understood by those skilled in the art that the present disclosure may be easily modified to another specific type without changing the technical spirit of an essential feature of the present disclosure. Thus, it is to be appreciated that the embodiments described above are intended to be illustrative in every sense, and not restrictive. For example, each component which is described as a singular form may be divided to be implemented and similarly, components which are described as a divided form may be combined to be implemented.

The scope of the present disclosure is represented by the claims to be described below rather than the detailed description, and it is to be interpreted that the meaning and scope of the claims and all the changes or modified forms derived from the equivalents thereof come within the scope of the present disclosure.

1. A volume-selective three-dimensional magnetic resonance image generating method, the method comprising steps of:
applying a frequency selective excitation pulse and a slab selection gradient magnetic field together to an object;
acquiring a signal generated from the object by the excitation pulse and the slab selection gradient magnetic field; and
generating a three-dimensional magnetic resonance image through encoding based on a readout gradient magnetic field maintaining vertically to the acquired signal and the slab selection gradient magnetic field.

2. The volume-selective three-dimensional magnetic resonance image generating method according to claim 1, wherein in the applying to an object, the slab selection gradient magnetic field which is determined to selectively spin-excite a predetermined volume region corresponding to a predetermined field of view is applied to the object.

3. The volume-selective three-dimensional magnetic resonance image generating method according to claim 2, wherein in the applying to an object, an asymmetric frequency selective excitation pulse or a symmetric frequency selective excitation pulse is applied.

4. The volume-selective three-dimensional magnetic resonance image generating method according to claim 3, wherein the asymmetric frequency selective excitation pulse includes a main lobe and includes an asymmetric sinc pulse from which a side lobe following the main lobe is removed.

5. The volume-selective three-dimensional magnetic resonance image generating method according to claim 4, wherein the acquiring of a signal includes:
acquiring an echo signal in a ramp period of the readout gradient magnetic field.

6. The volume-selective three-dimensional magnetic resonance image generating method according to claim 5, further comprising:
applying gridding interpolation to the acquired signal.

7. The volume-selective three-dimensional magnetic resonance image generating method according to claim 2, further comprising:
selecting a predetermined volume region based on a field of view corresponding to a region of the object to be captured; and
determining the slab selection gradient magnetic field corresponding to the selected volume region.

8. The volume-selective three-dimensional magnetic resonance image generating method according to claim 2, wherein in the applying to the object, a plurality of slab selection gradient magnetic fields having a slab selection direction corresponding to each axial direction of the three-dimensional coordinate system is applied to the object and
in the generating of a magnetic resonance image, the encoding is performed based on a plurality of readout gradient magnetic fields applied to each axial direction of the three-dimensional coordinate system so as to vertically correspond to the plurality of slab selection magnetic fields.

9. A volume-selective three-dimensional magnetic resonance image generating apparatus, comprising:
an excitation execution unit which applies a frequency selective excitation pulse and a slab selection gradient magnetic field together to an object;
a data acquisition unit which acquires a signal generated from the object by the excitation pulse and the slab selection gradient magnetic field; and
an encoding unit which generates a three-dimensional magnetic resonance image through encoding based on a readout gradient magnetic field maintaining vertically to the acquired signal and the slab selection gradient magnetic field.

10. The volume-selective three-dimensional magnetic resonance image generating apparatus according to claim 9, wherein the excitation execution unit applies the slab selection gradient magnetic field which is determined to selectively spin-excite a predetermined volume region corresponding to a predetermined field of view to the object.

11. The volume-selective three-dimensional magnetic resonance image generating apparatus according to claim 10, wherein the excitation execution unit applies an asymmetric frequency selective excitation pulse or a symmetric frequency selective excitation pulse and the asymmetric frequency selective excitation pulse includes a main lobe and includes an asymmetric sinc pulse from which a side lobe following the main lobe is removed.

12. The volume-selective three-dimensional magnetic resonance image generating apparatus according to claim 11, wherein the data acquisition unit acquires an echo signal in a ramp period of the readout gradient magnetic field.

13. The volume-selective three-dimensional magnetic resonance image generating apparatus according to claim 10, further comprising:
a region setting unit which selects a predetermined volume region based on a field of view corresponding to a region of the object to be captured and determines the slab selection gradient magnetic field corresponding to the selected volume region.

14. A non-transitory computer-readable storage medium storing instructions that, when executed by one or more processors, cause the one or more processors to perform the method of claim 1.
